(19) United States
(12) Patent Application Publication
(10) Pub. No.: US 2012/0128740 A1
(43) Pub. Date: May 24, 2012

(54) NANOSTRUCTURED SILDENAFIL BASE, ITS PHARMACEUTICALLY ACCEPTABLE SALTS AND CO-CRYSTALS, COMPOSITIONS OF THEM, PROCESS FOR THE PREPARATION THEREOF AND PHARMACEUTICAL COMPOSITIONS CONTAINING THEM

(76) Inventors: Genovéva Filipcsei, Budapest (HU); Zsolt Ötvös, Csongrad (HU); Katalin Pongrácz, Budapest (HU); Ferenc Darvas, Budapest (HU)

(21) Appl. No.: 13/378,640

(22) PCT Filed: Jun. 18, 2010
(86) PCT No.: PCT/HU10/00071

§ 371 Date: Feb. 13, 2012

(30) Foreign Application Priority Data
Jun. 19, 2009 (HU) P0900377
Apr. 19, 2010 (HU) P1000214

(51) Int. Cl. A61K 9/14; A61P 15/00; A61K 31/519; A61P 9/12; B82Y 5/00

(52) U.S. Cl. 424/400; 514/252.16; 977/773

(57) ABSTRACT

The present invention is directed to nanostructured (nanoparticulated) Sildenafil base, its pharmaceutically acceptable salts and co-crystals, compositions containing them, process for the preparation thereof and pharmaceutical compositions containing them. The nanoparticles of Sildenafil base, its pharmaceutically acceptable salts and co-crystals, compositions containing them according to the invention have an average particle size of less than about 500 nm. Sildenafil citrate is inhibiting cGMP specific phosphodiesterase type 5 (PDEV), an enzyme that regulates blood flow in the penis. The compositions of the invention are useful in the treatment of male or female sexual dysfunction and pulmonary arterial hypertension (PAH).

### FIELD OF THE INVENTION

The present invention is directed to nanostructured (nanoparticulated) Sildenafil base, its pharmaceutically acceptable salts and co-crystals, compositions of them, process for the preparation thereof and pharmaceutical compositions containing them.

The nanoparticles of Sildenafil base, its pharmaceutically acceptable salts and co-crystals, compositions of them according to the invention have an average particle size of less than about 500 nm. Sildenafil base and its salts, especially Sildenafil citrate is inhibiting cGMP specific phosphodiesterase type 5 (PDEV), an enzyme that regulates e.g. blood flow in the penis. The compositions of the invention are useful in the treatment of male or female sexual dysfunction and pulmonary arterial hypertension (PAH).

### BACKGROUND OF THE INVENTION

A. Background Regarding to Nanoparticle Formation/Production

Nanoparticles development for Pharmaceutical Applications deals with emerging new technologies for developing customized solutions for drug delivery systems. The drug delivery systems should positively impact the rate of absorption, distribution, metabolism, and excretion of the drug or other related chemical substances in the body. In addition, the drug delivery system should allow the drug to bind to its target receptor and influence that receptor's signaling and activity. Drug delivery materials should be compatible, easy to bind with a particular drug, and able to degrade into fragments after use that are either metabolized or driven out via normal excretory routes.

A different approach is to produce the active ingredient (API) in nanoparticulate form.

Nanoparticle compositions are described, for example, in U.S. Pat. No. 5,298,262, U.S. Pat. No. 5,318,767, U.S. Pat. No. 5,328,404 U.S. Pat. No. 5,336, U.S. Pat. No. 5,340,564 U.S. Pat. No. 5,466,440, U.S. Pat. No. 5,552,160, U.S. Pat. No. 5,560,931 U.S. Pat. No. 5,573,783, U.S. Pat. No. 5,593,657, US6,045,829, U.S. Pat. No. 6,264,922, U.S. Pat. No. 6,428,814 U.S. Pat. No. 6,592,903, U.S. Pat. No. 6,656,504, U.S. Pat. No. 6,976,647.

The API nanoparticles can be made using, for example, milling, homogenization, precipitation techniques, or supercritical fluid techniques, as is known in the art. Methods of making nanoparticulate compositions are also described in U.S. Pat. No. 5,718,388, U.S. Pat. No. 5,862,999, U.S. Pat. No. 5,665,331, U.S. Pat. No. 5,543,133, U.S. Pat. No. 5,534,270, U.S. Pat. No. 5,510,118, U.S. Pat. No. 5,470,583, U.S. Pat. No. 2009/0028948 and EP 1658053.

B. Background Regarding Sildenafil Citrate

Sildenafil citrate is designated chemically as 1-[[3-(6,7-dihydro-1-methyl-7-oxo-3-propyl-1H-pyrazolo[4,3-d]pyrimidin-5-yl)-4-ethoxyphenyl]sulfonyl]-4-methylpiperazine citrate and has the following structural formula:

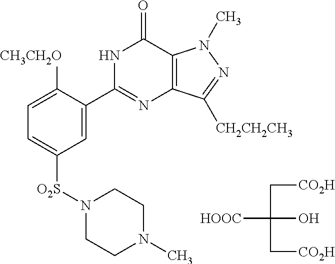

Sildenafil citrate is a white to off-white crystalline powder with a solubility of 3.5 mg/mL in water and a molecular weight of 666.7.

The method of its preparation was first described in U.S. Pat. No. 5,250,534. It is known that Sildenafil citrate can be used for treatment of erectile dysfunction as discussed in U.S. Pat. No. 6,469,012.

Sildenafil citrate is formulated as blue, film-coated rounded-diamond-shaped tablets equivalent to 25 mg, 50 mg and 100 mg of sildenafil citrate for oral administration and marketed under the name VIAGRA.

Additionally WO1999/021562 describes that PDEV inhibitors may be used for the treatment of female sexual dysfunction.

Pharmacological Properties

Oral Sildenafil citrate is rapidly absorbed, with peak plasma concentrations (cₘₐₓ) occurring within 1 hour. Absolute bioavailability is 41%. Food slows absorption but does not affect the area under the plasma sildenafil concentration-time curve (AUC). AUC and cₘₐₓ were dose-proportional over single sildenafil citrate doses from 1.25 to 200 mg.

Absorption and Distribution

Oral Sildenafil citrate is rapidly absorbed. Maximum observed plasma concentrations are reached within 30 to 120 minutes (median 60 minutes) of oral dosing in the fasted state. When VIAGRA is taken with a high fat meal, the rate of absorption is reduced, with a mean delay in tₘₐₓ of 60 minutes and a mean reduction in cₘₐₓ of 29%.

Side Effects

It is known that Sildenafil citrate taken orally may cause headache, flushing, visual effects, dyspersia. The use of oral Sildenafil citrate is contraindicated in subjects taking organic nitrates.

Because of the low solubility of Sildenafil citrate in water (3.5 mg/mL), the low (41%) bioavailability and the side effects, there is a need in the art to enhance the lipophilicity/bioavailability/increase the absorption/reduce the side effect/decrease the dosage/reduce the food effect in order to overcome these and other problems associated with the use of prior conventional Sildenafil citrate formulations. Moreover, these problems can be solved by surface modification to decrease the first pass effect or by modifying the metabolism of Sildenafil citrate. Beside the traditional formulation of Sildenafil citrate, the transdermal/topical application could increase the bioavailability and/or decrease the time which is needed to reach the desired effect of Sildenafil citrate. The present invention satisfies this need.

### DESCRIPTION OF THE INVENTION

The present invention describes the nanostructured (nanoparticulated) Sildenafil base, its pharmaceutically acceptable salts and co-crystals, compositions of them, with enhanced lipophilicity/bioavailability/increased absorption and dissolution rate/reduced side effect/decreased dosage/reduced food effect.

As exemplified in the examples below, not every combination of stabilizers will result in a stable nanoparticle formation. It was discovered, that stable Sildenafil nanoparticles can be made by continuous flow method, preferably by microfluidic based continuous flow method, using selected stabilizers. The expression Sildenafil is generally used for Sildenafil base, its pharmaceutically acceptable salts and co-crystals.

The invention comprises a nanostructured Sildenafil base, its pharmaceutically acceptable salts and co-crystals having an average particle size of less than about 500 nm.

Nanostructured Sildenafil base, its pharmaceutically acceptable salts and co-crystals according to the invention have an average particle size between 500 nm and 50 nm, preferably between 350 nm and 50 nm, preferably between 100 nm and 50 nm.

The invention further relates to a stable nanostructured Sildenafil composition comprising:
(a) nanostructured Sildenafil base, its pharmaceutically acceptable salts or co-crystals having an average particle size of less than about 500 nm;
(b) at least one anionic polyelectrolyte or a stabilizer or a mixture thereof or any additional stabilizer for steric and electrostatic stabilization.

The composition of the invention is prepared preferably in a continuous flow reactor, most preferable in a microfluidic based continuous flow reactor.

The compositions according to the invention contain Sildenafil base or its pharmaceutically acceptable salt or co-crystal having an average particle size of less than about 500 nm, preferably between 500 nm and 50 nm, preferably between 350 nm and 50 nm, preferably between 100 nm and 50 nm.

In the composition of the invention: (a) Sildenafil base or its pharmaceutically acceptable salt or co-crystal is present in an amount selected from the group consisting of from about 99.5% to about 0.001%, from about 95% to about 0.1%, and from about 90% to about 0.5%, by weight, based on the total combined weight of the Sildenafil base or its pharmaceutically acceptable salt or co-crystal and at least one stabilizer or polyelectrolyte, not including other excipients; (b) the stabilizer or polyelectrolyte is present in an amount selected from the group consisting of from about 0.5% to about 99.999% by weight, from about 5.0% to about 99.9% by weight, and from about 10% to about 99.5% by weight, based on the total combined dry weight of Sildenafil base or its pharmaceutically acceptable salt or co-crystal and at least one stabilizer, not including other excipients.

In the composition of the invention Sildenafil base or its pharmaceutically acceptable salt or co-crystal can be used in a phase selected from a crystalline phase, an amorphous phase, a semi-crystalline phase, a semi-amorphous phase and mixtures thereof in any polymorph form.

For the preparation of the composition of the invention anionic polyelectrolytes, preferably nucleic acids, proteins, teichoic acids, polypeptides, and polysaccharides (such as pectin, carrageenan, alginates, carboxymethyl cellulose (natural polylectrolytes)) and poly(sodium styrene sulfonate) (PSS) and poly (acrylic-acid) and its derivatives cross-linked with allyl esters or sucrose or pentaerythriol (e.g.: Carbopol 2623, Carbopol 971P, Carbopol 980, Pemulen TR1, Pemulen TR2), poly(meth)acrylate-based polymers and copolymers (Eudargit®) (synthetic); non-ionic stabilizes preferably poly(vinyl-pyrrolidone), poly(2-ethyl-2-oxazoline), poly(methyl vinyl ether), polyvinyl alcohol, acetic acid ethenyl ester polymer with 1-ethenyl-2-pyrrolidinone (PVPNA copolymers), poly(ethylene-glycol) and its derivatives (e.g.: PEG 2000, 6000, 35 000), ethylene oxide and propylene oxide block copolymers and its derivatives (e.g.: Pluronic 10 500, 6100, 6800), and polyoxyethylene sorbitan fatty acid esters (e.g., the commercially available Tween® products such as e.g., Tween® 20 and Tween® 80 (ICI Speciality Chemicals)); and as additional stabilizer preferably hydroxyl-propyl-cellulose derivatives, sodium lauryl sulfate, sodium dodecyl benzene sulfonate, tocopheryl polyethylene glycol succinates, polyethoxylated castor oils and its derivatives, any cationic stabilizers, preferably lauryl trimethyl ammonium chloride, alkylbenzyl methyl ammonium chloride, alkylbenzyl dimethyl ammonium bromide, benzyl trimethylammonium bromide, benzalkonium chloride, hexadecyltrimethylammonium bromide can be used.

Advantages of the composition of the invention include, but are not limited to: (1) smaller tablet or other solid dosage form size and beneficial transdermal/topical application; (2) lower doses of drug required to obtain the same pharmacological effect as compared to conventional forms of Sildenafil citrate; (3) increased bioavailability as compared to conventional forms of Sildenafil citrate; (4) improved pharmacokinetic profiles; (5) an increased rate of dissolution for Sildenafil citrate nanoparticles as compared to conventional forms of the same active compound; (6) modified metabolism of Sildenafil citrate nanoparticles. Another aspect of the invention is a process for the preparation of nanostructured Sildenafil base or its pharmaceutically acceptable salts or co-crystals comprising mixing an appropriate solution of Sildenafil base or its pharmaceutically acceptable salt or co-crystal with a solution of one or more stabilizers or polyelectrolytes or a mixture thereof, if desired in the presence of a pharmaceutically acceptable acid or base in a continuous flow reactor.

Preferably the process for the preparation of the composition of the invention is carried out by (1) dissolving Sildenafil base or its pharmaceutically acceptable salt or co-crystal and optionally one or more stabilizer or polyelectrolytes or a mixture thereof in a suitable solvent; (2) adding the formulation from step (1) to a solution comprising one or more polyelectrolytes or stabilizers or a mixture thereof, if desired in the presence of a pharmaceutically acceptable acid or base; and (3) precipitating the formulation from step (2).

Preferably the process for the preparation of the composition of the invention is carried out by (1) dissolving Sildenafil base or its pharmaceutically acceptable salt or co-crystal and one or more stabilizers in a suitable solvent;(2) adding the formulation from step (1) to a solution from step (1) to a solution comprising one or more polyelectrolytes or stabilizers or a mixture thereof, if desired in the presence of a pharmaceutically acceptable acid or base; and (3) precipitating the formulation from step (2).

Another preferred embodiment of the invention is where the process for the preparation of the composition is carried out by (1) dissolving Sildenafil base or its pharmaceutically acceptable salt or co-crystal and one or more stabilizers in a suitable solvent; (2) adding the formulation from step (1) to a solution comprising a pharmaceutically acceptable acid or base; and (3) precipitating the formulation from step (2).

The process is carried out by (a) using two different solvents miscible with each other, where Sildenafil base or its pharmaceutically acceptable salt or co-crystal is soluble only in one of them, or (b) using the same solvent in the two steps, where the polyelectrolyte complex of Sildenafil base or its pharmaceutically acceptable salt or co-crystal forms nanostructured particles, practically, with the restriction that the applied polyelectrolyte, stabilizer(s) is soluble in the solvents used.

As a continuous flow reactor preferable a microfluidics based continuous flow reactor, described in the publication Microfluid Nanofluid DOI 10.1007/s10404-008-0257-9 by I. Hornyak, B. Borcsek and F. Darvas, is used.

If in the process of the invention two different solvents are used for the chemical precipitation they have to be miscible with each other, where Sildenafil is soluble only in one of them. Such solvents may be dimethyl-sulfoxyde, ethanol, i-propanol, tetrahydrofuran, acetone, methyl-ethyl-ketone, dimethyl-formamide, diethylene-glycol-ethyl-ether, pyridine preferably. For the polyelectrolyte complexation water based solution can be used, preferably.

The particle size of the nanostructured Sildenafil base or its pharmaceutically acceptable salts or co-crystals may be influenced by the solvents used, the flow rate and the Sildenafil-stabilizer ratio.

Another aspect of the invention is directed to the good/instantaneous redispersibility of solid nanosized form of Sildenafil in biologically relevant mediums, e.g.; physiological saline solution, pH=2.5 HCl solution.

Another aspect of the invention is a pharmaceutical composition comprising a stable nanostructured Sildenafil base or its pharmaceutically acceptable salts or co-crystals, or composition of them according to the invention and optionally pharmaceutically acceptable auxiliary materials.

The pharmaceutical composition of the invention can be formulated: (a) for administration selected from the group consisting of oral, pulmonary, rectal, colonic, parenteral, intracisternal, intravaginal, intraperitoneal, ocular, otic, local, buccal, nasal, and topical administration; (b) into a dosage form selected from the group consisting of liquid dispersions, gels, aerosols, ointments, creams, lyophilized formulations, buccal films, tablets, capsules; (c) into a dosage form selected from the group consisting of controlled release formulations, fast melt formulations, delayed release formulations, extended release formulations, pulsatile release formulations, and mixed immediate release and controlled release formulations; or (d) any combination of (a), (b), and (c).

The compositions can be formulated by adding different types of excipients for oral(solid, liquid), vaginal, rectal, local (powders, ointments, gels, or drops), or topical administration, and the like.

The most preferred dosage form of the invention is the buccal film and gel dosage form, although any pharmaceutically acceptable dosage form can be utilized.

For oral delivery into the human body nanoparticles can be also administered as their aqueous dispersion as the final dosage form. This is a way of delivery without further processing after nanoparticle formation. However, poor stability of the drug or polymer in an aqueous environment or poor taste of the drug may require the incorporation of the colloidal particles into solid dosage forms, i.e. into capsules and tablets.

Alternatively, the aqueous dispersion of the colloidal particles can be incorporated into the solid dosage form as a liquid, for example by granulation of suitable fillers with the colloidal dispersion to form a granulation. Such granules can subsequently be filled into capsules or be compressed into tablets. Alternatively, through layering of the dispersion onto e.g. sugar-pellets as carriers in a fluidized bed a solid form for nanoparticles can be. These ways of manufacturing tablet cores, or granules or pellets can potentially by followed by a coating step to reveal a film-coated tablet or film coated granules in a capsule as the final dosage form.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols(propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

Solid dosage forms for oral administration include, but are not limited to, capsules, tablets, pills, film coated tablets, pills, powders, and granules. In such solid dosage forms, the active agent is admixed with at least one of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite (j) film coating materials as methacrylic acid/methacrylate esters, polyvinylacetate phthalate, cellulose acetate phthalate, hydroxypropylmethylcellulose, hydroxypropylethylcellulose, ethylcellulose, methylcellulose; and (i) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, solutions, suspensions, syrups, and elixirs. In addition to the Sildenafil base or its pharmaceutically acceptable salts or co-crystals, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

The pharmaceutical compositions of the invention show enhanced lipophilicity/bioavailability/increased absorption and dissolution rate/reduced side effect, they can be used in a decreased dosage in the treatment of male and female sexual dysfunction and pulmonary arterial hypertension, as compared to conventional Sildenafil citrate form.

The present invention is also directed to methods of treating erectile dysfunction, female sexual dysfunction and pulmonary arterial hypertension (PAH) using the novel Sildenafil nanoparticles disclosed herein.

### A. Preferred Characteristics of the Sildenafil Nanoparticles of the Invention

### 1. Increased Bioavailability

The nanostructured Sildenafil base or its pharmaceutically acceptable salts or co-crystals compositions of the invention are proposed to exhibit increased bioavailability, faster onset of action, reduced food effect and require smaller doses as compared to prior known, conventional Sildenafil citrate formulations.

### EXAMPLE 1

Comparison of Relative Bioavailability of Two Sildenafil Formulation in Dogs

The aim of this study was to investigate the relative bioavailability of the buccal test formulation (nanosized sildenafil composition) of example 9 and the reference Viagra tablet administered orally under fed condition.

Animals

The Beagle dog is suitable non-rodent species for pharmacokinetic studies and is acceptable to regulatory authorities. The dog is readily available, easy to handle, house and dose and suitable for investigation of the whole plasma level curve in each individual animal.

The systemic exposure was investigated in the same three dogs for both the test and reference items. This group size is optimal for pharmacokinetic studies in large animals.

The animals received ssniff Hd-H diet for dogs produced by Ssniff, Spezialdiaten GmbH. The food was offered daily 300 g/dog approximately at the same time. The next morning the remaining food was taken away.

Before the administrations, the animals were fasted overnight and on the treatment days 1 hour prior to the administrations the animals received approximately 150 g standard diet. The other 150 g food was offered at approximately 4 hours after the administration.

Administration

Relative bioavailability of the sildenafil formulations were investigated in a single dose (25 mg/dog is a non-toxic oral dose), two period study. Buccal administration was performed by placing the solid nanostructured formula on the mucosal surface of the front of the mouth and holding the dogs' mouth for 15 minutes to allow the absorption of the formulation. The formulation was immediately dissolved in the mouth and by 15 minutes there were no remnants of the dose administered.

Blood Collection and Plasma Separation

For determination of plasma levels of sildenafil approximately 3 ml of blood was collected in plastic vials with lithium heparin as anticoagulant. The time points of blood collection were the followings in both periods: pre-dose (0 min), 15 min, 30 min, 45 min, 1 h, 1.5 h, 2 h, 3 h, 6 h, 9 h, 12 h, 24 h and 48 h after dosing.

Blood was withdrawn the v. cephalica antebrachii or v. saphena with sterile, disposable needles. After sampling, the blood was kept cooled on crushed ice until centrifugation. Plasma samples were prepared by centrifugation of the blood at 2,000 g for 10 minutes at 4° C. within 60 minutes after blood sampling. The separated plasma (approx. 1 ml) was transferred into Eppendorf tubes. Plasma samples were immediately frozen and stored in deep-freezer (−20±5° C.) until analysis.

The concentrations of Sildenafil were determined using a reliable chromatographic bioanalytical method.

Pharmacokinetic Evaluation

The pharmacokinetic evaluation was performed at the Analytical Department of ATRC using WinNonlin Professional Version 4.0.1 software (Pharsight Corporation, USA). The individual plasma levels versus time curves were evaluated using a non compartmental method.

Results

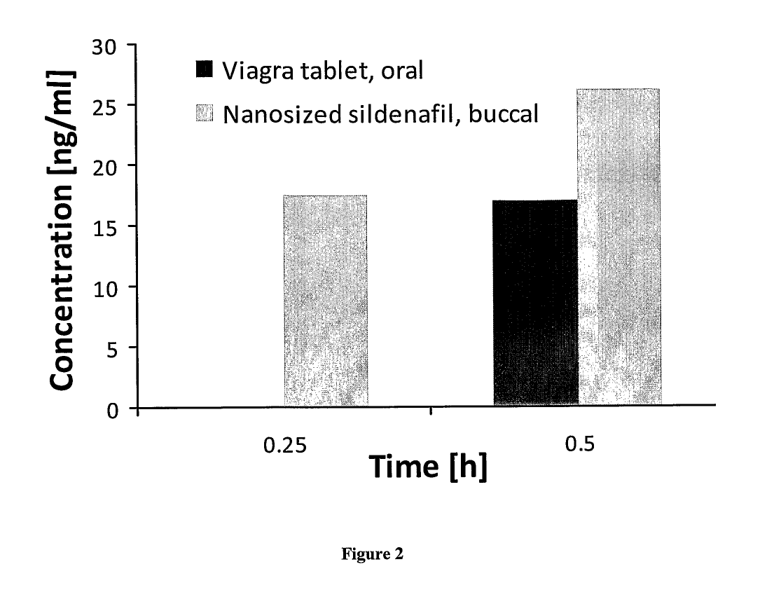

Both oral administration of Viagra tablets and buccal administration of nanostructured Sildenafil (25 mg dose in both cases) resulted in a detectable serum concentration exhibiting a biphasic profile in the 15 min-48 h interval. The buccal absorption of nanostructured Sildenafil took place with a faster onset. Plasma concentrations for the buccal administration could be detected as early as 15 minutes after administration, while no plasma Sildenafil could be detected after the administration of the tablet at this time point. Furthermore, plasma Sildenafil concentrations were still higher for the buccal formulation at 30 min (FIG. 2).

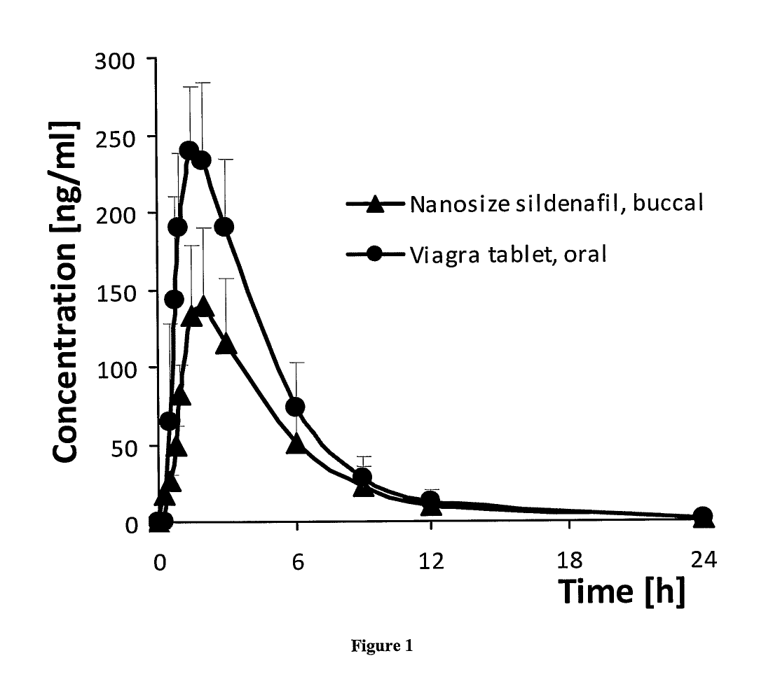

Area under the curve for the whole study period (0-48 h) (AUCₗₐₛₜ), cₘₐₓ and tₘₐₓ were calculated from the curves and relative bioavailability (Fᵣₑₜ) of the nanosized formulation compared to the marketed drug was determined (Table 1). The two formulations and routes of administration had very similar pharmacokinetic profile, with practically identical tₘₐₓ values. Exposure (AUC) and cₘₐₓ values are lower for the buccal administration with 62% relative bioavailability compared to the oral administration of the marketed drug (FIG. 1, Table 1).

Altogether, the buccal administration of nanostructured Sildenafil results in faster appearance of Sildenafil in plasma and similar kinetic profile with lower bioavailability. Furthermore, no fed/fasted effect can be expected in case of the buccal route.

FIG. 1: Serum concentrations of Sildenafil at early time points after oral administration of the reference tablet and buccal administration of 25 mg/kg nanostructured Sildenafil

FIG. 2: Serum concentrations of Sildenafil after oral administration of the reference tablet and buccal administration of 25 mg/kg nanostructured Sildenafil

Table 1: Main pharmacokinetic parameters of oral Viagra and buccal Sildenafil nanoformula administration

### 2. Solubility and Dissolution Profiles of the Nanostructured Sildenafil Base or its Pharmaceutically Acceptable Salts or Co-Crystals Compositions of the Invention

The nanostructured Sildenafil base or its pharmaceutically acceptable salts or co-crystals compositions of the invention have increased solubility and dissolution profile due to the decreased particle size and nanostructured particle formation. Rapid dissolution of an administered active agent is preferable, as faster dissolution generally leads to faster onset of action and greater bioavailability.

### EXAMPLE 2

Determination of Cₘₐₓ

The solubility of nanostructured Sildenafil Citrate of example 9 compared to the reference API was determined in distillate water by UV-VIS measurements (Helios Alfa UV spectrophotometer) at 292 nm wavelength and room temperature. The redispersed sample was filtered by 0.45 μm disposable syringe filter. In order to check the nanoparticle presence in the solution, it was irradiated by red laser pointer operating at 670 nm wavelength. If no scattering was observed the filtration was successful, the solution did not contain nanoparticles.

The solubility of the nanostructured Sildenafil Citrate is 24.5 mg/mL which is 6.8 times higher than the solubility of Sildenafil Citrate in distillate water.

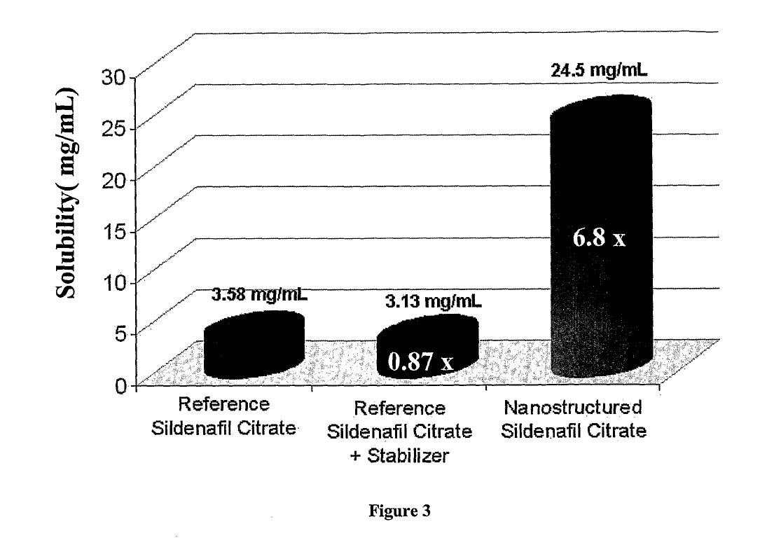

FIG. 3: Solubility enhancement of Sildenafil Citrate by nanoformulation

### EXAMPLE 3

Increased Dissolution Rate of Nanosized Composition

The dissolution rate of nanostructured Sildenafil Citrate of example 9 compared to the reference API was determined in distillate water by UV-VIS measurements (Agilent 8453) at 292 nm wavelength and room temperature. 37.5 mg reference Sildenafil citrate was suspended in 1.5 mL distilled water, while 40.76 mg nanostructured Sildenafil Citrate powder containing 37.5 mg Sildenafil Citrate was suspended in 1.5 mL distillate water. The suspension was stirred for 1 second, 1, 3 and 5 minutes and then was filtered by 0.45 μm disposable syringe filter. In order to check the nanoparticle presence in the solution, it was irradiated by red laser pointer operating at 670 nm wavelength. If no scattering was observed the filtration was successful, the solution did not contain nanoparticles.

The results showed significant difference. In the first second the amount of the dissolved Sildenafil Citrate from the nanostructured Sildenafil Citrate was 6.19 times higher compared to the reference.

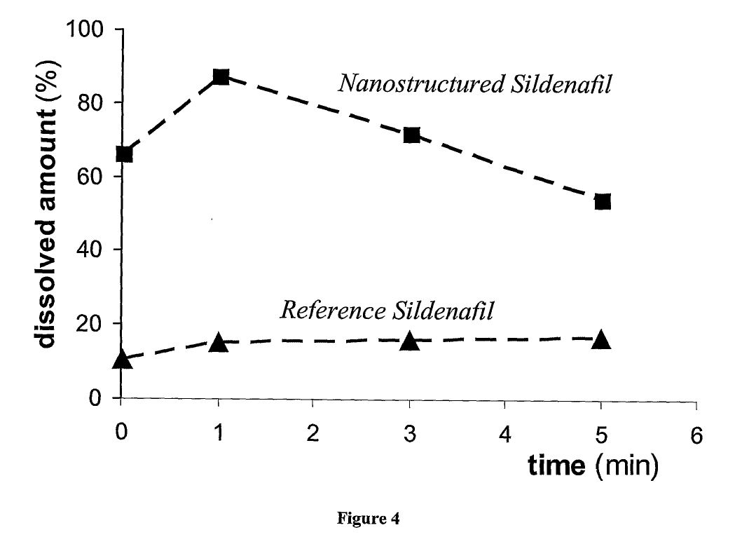

FIG. 4: Dissolution profile of nanostructured Sildenafil Citrate compared to the reference Sildenafil Citrate

### 3. Crystallographic Structure of Nanostructured Sildenafil Base or its Pharmaceutically Acceptable Salts or Co-Crystals Compositions of the Invention

The chemical stability of solid drugs is affected by the crystalline state of the drug. Many drug substances exhibit polymorphism. Each crystalline state has different chemical reactivity. The stability of drugs in their amorphous form is generally lower than that of drugs in their crystalline form, because of the higher free-energy level of the amorphous state.

Decreased chemical stability of solid drugs brought about by mechanical stresses such as grinding is to a change in crystalline state.

The chemical stability of solid drugs is also affected by the crystalline state of the drug through differences in surface area. For reaction that proceeds on the solid surface of drug, an increase in the surface area can increase the amount of drug participating in the reaction.

### EXAMPLE 4

Crystallographic Structure Determination

Stable amorphous/partly crystalline/crystalline/polymorph Sildenafil base or its pharmaceutically acceptable salts or co-crystals compositions of the invention shows significantly enhanced solubility due to its increased surface area when compared to a crystalline reference.

The structure of the Sildenafil citrate nanoparticles of example 9 prepared by polylectrolyte complex formation, using Carbopol 971 (acrylic-acid polymer cross-linked with allyl ethers) was investigated by X-ray diffraction analysis (Philips PW1050/1870 RTG powder-diffractometer). The measurements showed that the nanostructured Sildenafil citrate compositions are partly crystalline. The wide reflection between 15 and 20 20 values indicates the amorphous structure of the Carbopol 971. The characteristic reflections of the crystalline Sildenafil citrate can be found on the XRD diffractogram of nanosized Sildenafil citrate, but with lower intensity. This showed that the nanonization resulted in a partly crystalline Sildenafil citrate form. The X-ray diffractograms are demonstrated in FIG. 6.

Based on the diffractograms, it can be concluded, that the particle size of the reference Sildenafil citrate is 1-2 μm, however the nanosized Sildenafil citrate has the particle size less than 100 nm.

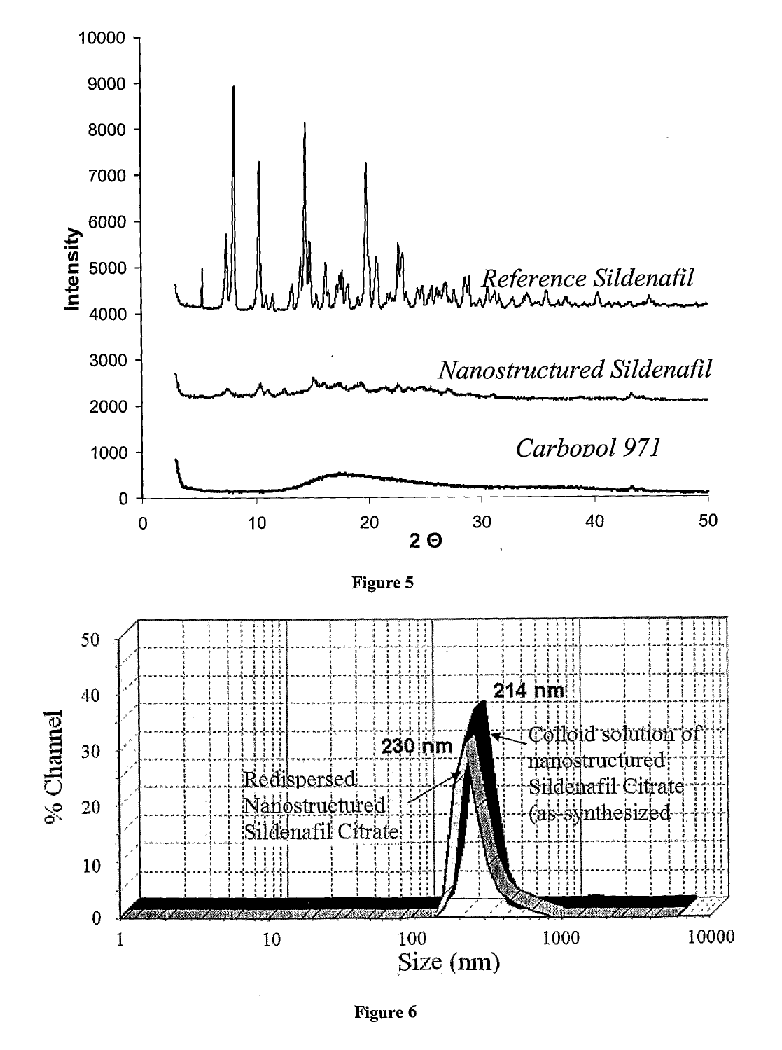

FIG. 5: X-ray diffractograms of reference Sildenafil citrate, nanostructured Sildenafil citrate of the invention and Carbopol 971

### 4. Redispersibility Profiles of the Nanostructured Sildenafil Base or its Pharmaceutically Acceptable Salts or Co-Crystals Compositions of the Invention

An additional feature of the nanostructured Sildenafil base or its pharmaceutically acceptable salts or co-crystals compositions of the present invention is that the dried nanoparticles stabilized by surfactant(s) can be redispersed instantaneously or by the addition of traditional redispersants such as mannitol, sucrose.

### EXAMPLE 5

Redispersibility Test

Redispersibility test was performed to determine the solubility of the nanostructured Sildenafil Citrate of example 9 in distillate water. 15 mg freeze dried nanostructured Sildenafil Citrate and 50 mg Mannitol were redispersed in 10 mL distillate water under vigorous stirring. The particles size of the redispersed sample was detected by DLS method (Nanotrac instrument, Mictrotrac Co., USA).

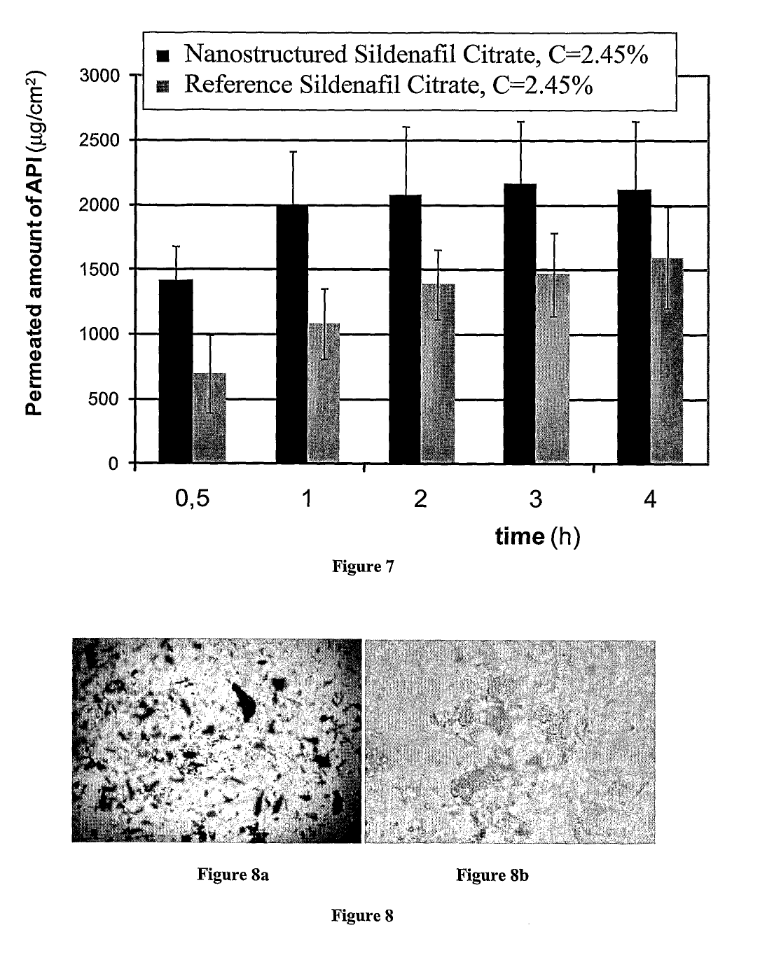

The main particle size of redispersed nanostructure Sildenafil Citrate (intensity-based average) is d=230 nm, while d(90) value is 368 nm demonstarted in FIG. 7.

The significant benefit which can be obtained by nanoformulation is that the Sildenafil citrate nanoparticles of the present invention can be redispersed after the drying/solid formulation procedure having similar average particle size. Having the similar average particles size after the redispersion, the dosage form cannot loose the benefits afforded by the nanoparticle formation. A nano-size suitable for the present invention is an average particle size of less than about 290 nm.

FIG. 6: Size and size distribution of the Sildenafil citrate nanoparticles before and after the redispersion

### 5. Enhanced Lipophilicity to Increase the Absorption and Permeability Profiles of the Nanostructured Sildenafil Base or its Pharmaceutically Acceptable Salts or Co-Crystals Compositions of the Invention

Due to the phospholipidic nature of cell membranes, a certain degree of lipophilicity is often a requirement for the drug compound, not only to be absorbed through the intestinal wall following oral administration but possibly also to exert its pharmacological action in the target tissue. (F. Kesisoglou et al./Advanced Drug Delivery Reviews 59 (2007) 631-644).

The lipophilicity of Sildenafil can be increased by using lipophilic stabilizer and/or stabilizers having lipophilic side groups on the polymeric backbone and/or amphiphil stabilizers during the nano precipitation. Due to the lipophilic nature or lipophilic side groups of the applied stabilizer, not only lipophilicity, but absorption and permeability of the Sildenafil nanoparticles of the present invention can be increased.

For example using Chitosan, it can increase the paracellular permeability of intestinal epithelia which attributed to the transmucosal absorption enhancement.

Most amphiphilic copolymers employed for drug delivery purposes contain either a polyester or a poly(amino acid)-derivative as the hydrophobic segment. Most of the polyethers of pharmaceutical interest belong to the poloxamer family, i.e. block-copolymers of polypropylene glycol and polyethylene glycol.

### EXAMPLE 6

Comparative in vitro Permeability Tests

In vitro experiments were performed in vertical Franz-diffusion cell equipped with an autosampler (Hanson Microette™ Topical & Transdermal Diffusion Cell System, Hanson Research Corporation). During the permeability experiments 300 μl Sildenafil Citrate solution was placed to the Prorafil membrane as donor phase. The effective diffusion surface area was 1.767 cm². The acceptor phase was distillate water in all cases. All measurements were performed at 37° C. and 6 parallel samples were investigated.

For the permeability test 24.5 mg/mL Sildenafil Citrate reference suspension and 24.5 mg/mL nanostructured Sildenafil Citrate of example 9 solution were prepared and used after filtration. In both cases the permeated amount was detected as it is seen on FIG. 8.

The results showed significant difference. In the first 30 minutes the amount of the penetrated Sildenafil Citrate from the donor solution prepared from the nanostructured Sildenafil Citrate was 390% higher compared to the reference.

FIG. 7: Permeability enhancement using nanostructured Sildenafil Citrate

### 6. Faster Surface Wetting Profiles of the Nanostructured Sildenafil Base or its Pharmaceutically Acceptable Salts or Co-Crystals Compositions of the Invention

For the Sildenafil Citrate to dissolve, its surface has first to be wetted by the surrounding fluid. The nanosized partly crystalline forms possess a chemically randomized surface which expresses hydrophobic and hydrophilic interactions due to the nature of the stabilizer/(s) and active pharmaceutical ingredient, which can lead to improved wettability. If the surface of the Sildenafil citrate nanoparticles of the invention is functionalized by hydrophilic groups/stabilizer(s), a higher degree of hydrophility causes faster surface wetting and faster dissolution compared to the original crystalline form. This advanced property of the Sildenafil citrate nanoparticles of the present invention is supported by the results of the redispersibility test. Due to the bigger surface area of the nanostructured Sildenafil citrate particles and the hydrophilic groups of the stabilizer(s) the surface wetting is faster than that of the crystalline form's.

### EXAMPLE 7

Visual Observation of Nanostructured Sildenafil Base or its Pharmaceutically Acceptable Salts or Co-Crystals Wettability

Wettability of nanostructured Sildenafil Citrate particles was investigated in distilled water and was visualized by stereomicroscope equipped with CCD camera. 0.1 mg reference and nanostructure Sildenafil powder was placed to the slide and then one drop of distillate water was added to the powder. Nanostructured Sildenafil Citrate powder started to swell immediately, its wetting was complete, while the reference Sildenafil Citrate particles stayed in their aggregated state as it is demonstrated in FIG. 8.

FIG. 8: Wettability of reference Sildenafil Citrate (a) and nanostructured Sildenafil Citrate of example 9 (b) observed by stereomicroscope in 40× magnification

B. Compositions

The invention provides nanosized Sildenafil base, its pharmaceutically acceptable salts and co-crystals nanostructured particle formations comprising at least one stabilizer to stabilize them sterically and/or electrostatically.

The stabilizers preferably are associated or interacted with the Sildenafil base, its pharmaceutically acceptable salts and co-crystals but do not chemically react with them or themselves.

The nanoparticles of Sildenafil base, its pharmaceutically acceptable salts and co-crystals of the invention can be formed by complexation using biocompatible or biodegradable polyelectrolyte or can be prepared by solvent-antisolvent precipitation methods using stabilizer(s). The stability of the prepared colloid solution of nanosized Sildenafil citrate can be increased by combination of the complexation with steric or electrostatic particle stabilization. Moreover, using additional stabilizer the particle size of Sildenafil base, its pharmaceutically acceptable salts and co-crystals of the invention can be decreased and controlled.

Particle Size of Sildenafil Base, its Pharmaceutically Acceptable Salts and Co-Crystals Nanoparticles

The invention contains Sildenafil base, its pharmaceutically acceptable salts and co-crystals nanoparticles, which have an average particle size of less than about 500 nm as measured by dynamic light scattering method.

By “an average particle size of less than about 500 nm” it is meant that at least 50% of the Sildenafil base, its pharmaceutically acceptable salts and co-crystals nanoparticles have a particle size of less than the average, by number/intensity, i.e., less than about 500 nm, etc., when measured by the above-noted technique.

### EXAMPLE 8

Nanostructured Sildenafil Production

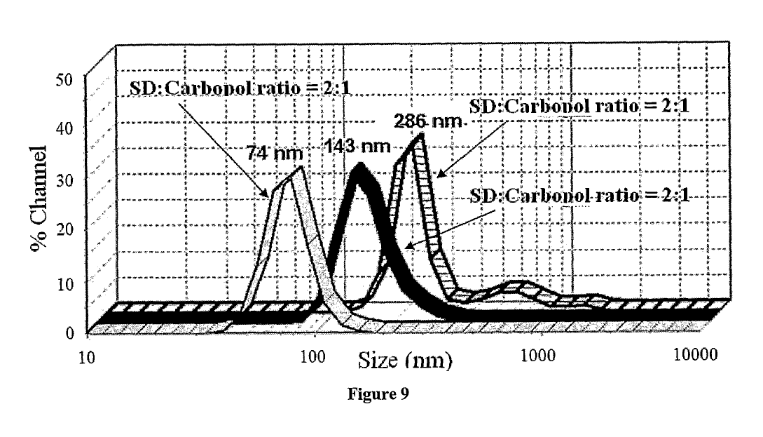

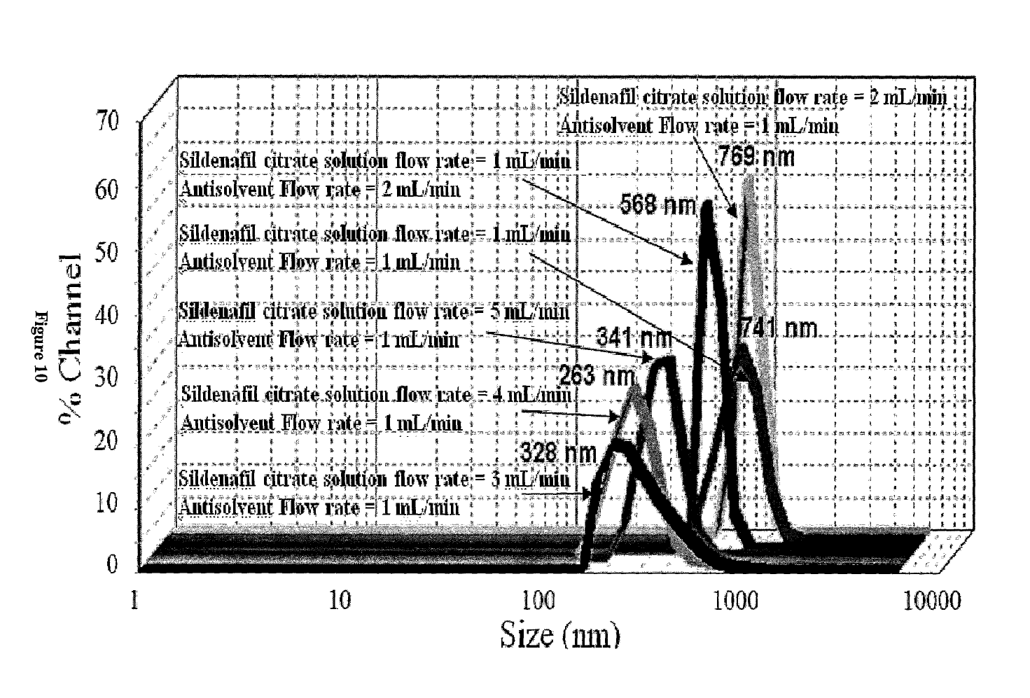

During the experiments Sildenafil citrate nanoparticles were prepared in a microfluidic based continuous flow reactor. As a starting solution, 250 mg Sildenafil citrate (SD) dissolved in 100 mL distilled water was used. The prepared solution was passed into the reactor unit with 3 mL/min flow rate using a feeding unit. Meanwhile, using a second feeding unit, a solution of 2.5-25 mg Carbopol 971 (CD) (Lubrisol) dissolved in 100 mL distilled water was passed into a mixing unit with 1 mL/min flow rate, where it was mixed with the solution containing Sildenafil Citrate coming from the first reactor unit. The nanoparticles are continuously produced at atmospheric pressure due to the polyelectrolyte complex formation by Carbopol 971 solution passed into the mixing unit. The produced colloidal solution driven through the second reactor unit getting to the dynamic light scattering unit (Nanotrac) integrated to the device, which can detect the particle size of the obtained nanoparticle continuously. The size of the nanoparticles can be controlled in wide range by changing the flow rates; pressure and the amount of the applied Carbopol 971 (see FIG. 9.). The particles size of the Sildenafil citrate particle was 74 nm in the best case (see Table 2). Changing the flow rates the particles size can be varied from 70 up to 500 nm.

FIG. 9: Particle size and size distribution of Sildenafil citrate nanoparticles using different API:polyelectrolyte ratio

Table 2: Effect on the flow rates on the particle size of Sildenafil citrate

### EXAMPLE 9

Nanostructured Sildenafil citrate production

During the experiments Sildenafil citrate nanoparticles were prepared in a microfluidic based continuous flow reactor. As a starting solution, 200 mg Sildenafil citrate (SD) dissolved in 60 mL distilled water was used. The prepared solution was passed into the reactor unit with 4 mL/min flow rate using a feeding unit. Meanwhile, using a second feeding unit, a solution of 50 mg sodium dodecylbenzene sulfonate (SDBS) dissolved in 100 mL distilled water was passed into a mixing unit with 1 mL/min flow rate, where it was mixed with the solution containing Sildenafil Citrate coming from the first reactor unit. The nanoparticles are continuously produced at atmospheric pressure due to the precipitating effect of SDBS solution passed into the mixing unit. The produced colloidal solution driven through the second reactor unit getting to the dynamic light scattering unit (Nanotrac) integrated to the device, which can detect the particle size of the obtained nanoparticle continuously. The size of the nanoparticles can be controlled in wide range by changing the flow rates (see FIG. 10.). The particles size of the Sildenafil citrate particle was 263 nm in the best case (see Table 3). Changing the flow rates the particles size can be varied from 263 up to 769 nm.

FIG. 10: Particle size and size distribution of Sildenafil citrate nanoparticles using different API:antisolvent ratio

Table 3: Effect on the flow rates on the particle size of Sildenafil citrate

### EXAMPLE 10

Nanostructured Sildenafil Base Production

During the experiments Sildenafil base nanoparticles were prepared in a microfluidic based continuous flow reactor. As a starting solution, 100-300 mg Sildenafil citrate (SD) and 60-1000 mg polyvinyl alcohol (PVA, Mw=30,000-70,000) dissolved in 60 mL distilled water was used. The prepared solution was passed into the reactor unit with 1-10 mL/min flow rate using a feeding unit. Meanwhile, using a second feeding unit, 0.001-0.1 M sodium hydroxide (NaOH) solution was passed into a mixing unit with 1-10 mL/min flow rate, where it was mixed with the solution containing Sildenafil Citrate coming from the first reactor unit. The nanoparticles are continuously produced at atmospheric pressure due to the precipitating effect of NaOH solution passed into the mixing unit. The produced colloidal solution driven through the second reactor unit getting to the dynamic light scattering unit (Nanotrac) integrated to the device, which can detect the particle size of the obtained nanoparticle continuously. The size of the nanoparticles can be controlled in wide range by changing the flow rates. The particles size of the Sildenafil base particle was 349 nm in the best case (see FIG. 8). Changing the flow rates the particles size can be varied.

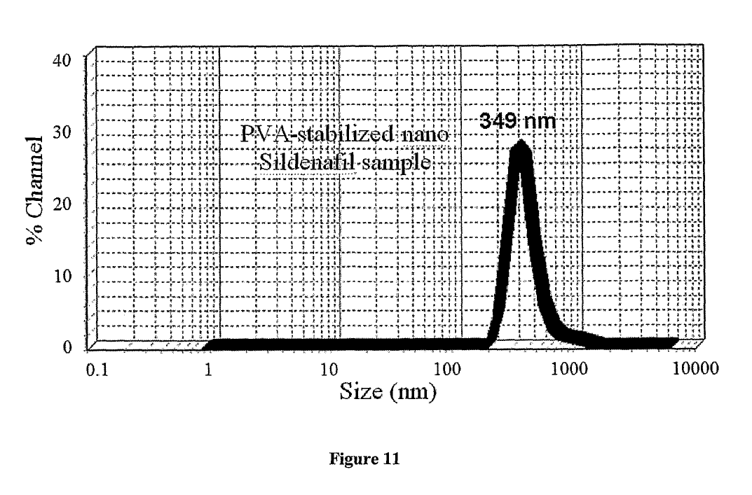

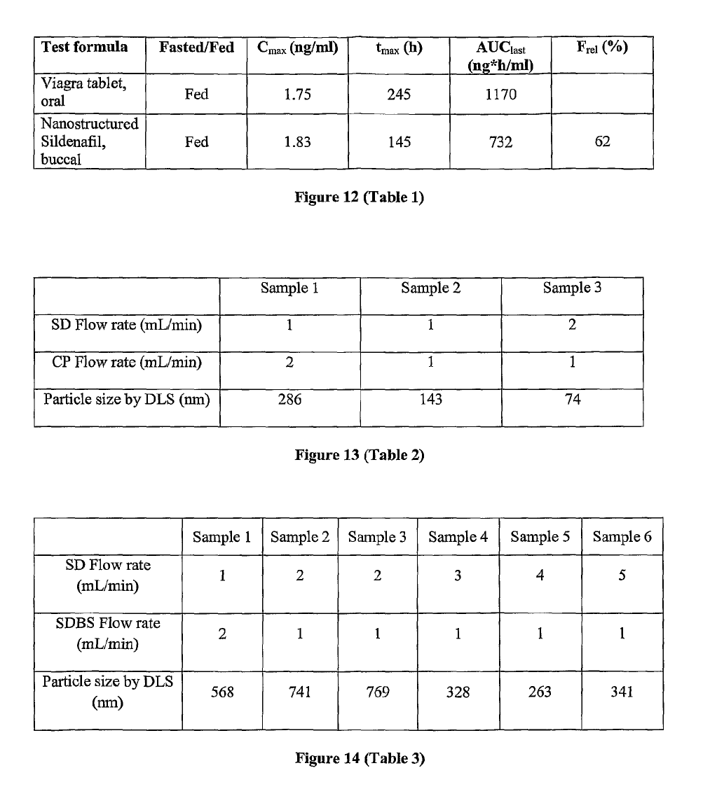

FIG. 11: Particle size and size distribution of Sildenafil base nanoparticles using different API:antisolvent ratio

### EXAMPLE 11

Sildenafil Base, its Pharmaceutically Acceptable Salts and Co-Crystals Nanoparticles Loaded Buccal Film Formulation

Films were made using hydroxypropylmethyl cellulose:polyethylene glycol 400:carbopol 934P in 0.3:1.0:0.7 ratio. A total of 1% w/v polymeric solution were allowed to stir for 6 h and stand overnight to remove all the air bubbles entrapped. Nanostructured Sildenafil Citrate was added and the solution was casted onto a petri dish and dried in the oven at 60° C. until completely dry. The film was carefully removed from the petri dish, checked for any imperfections and cut according to the size required for testing.

1-33. (canceled)

34. A stable nanostructured Sildenafil composition comprising:
(a) nanostructured Sildenafil base or its pharmaceutically acceptable salts or co-crystals having an average particle size of less than about 500 nm;
(b) at least one anionic polyelectrolyte or a non-ionic stabilizer or a mixture thereof or additional stabilizer,
wherein the composition is prepared in a continuous flow reactor.

35. The composition according to claim 34, wherein the continuous flow reactor is a microfluidic based continuous flow reactor.

36. The composition according to claim 34, wherein the average particle size is between 500 nm and 50 nm.

37. The composition according to claim 34, wherein the average particle size is between 350 nm and 50 nm.

38. The composition according to claim 34, wherein the average particle size is between 100 nm and 50 nm.

39. The composition according to claim 34 wherein the polyelectrolyte is selected from the group of nucleic acids, proteins, teichoic acids, polypeptides and polysaccharides and poly(sodium styrene sulfonate) and poly(meth)acrylate-based polymers and copolymers (synthetic), poly(acrylic-acid) and its derivatives cross-linked with allyl esters or sucrose or pentaerythriol.

40. The composition according to claim 34 wherein the non-ionic stabilizer is selected from the group of poly(vinyl-pyrrolidone), poly(2-ethyl-2-oxazoline), poly(methyl vinyl ether), polyvinyl alcohol, acetic acid ethenyl ester polymers with 1-ethenyl-2-pyrrolidinone (PVP/VA copolymers), poly(ethylene-glycol) and its derivatives, ethylene oxide and propylene oxide block copolymers and its derivatives, and polyoxyethylene sorbitan fatty acid esters.

41. The composition according to claim 34, where the composition contains additional stabilizer which is hydroxyl-propyl-cellulose derivatives, sodium lauryl sulfate, sodium dodecyl benzene sulfonate, tocopheryl polyethylene glycol succinates, polyethoxylated castor oils and its derivatives, any cationic stabilizers, preferably lauryl trimethyl ammonium chloride, alkylbenzyl methyl ammonium chloride, alkylbenzyl dimethyl ammonium bromide, benzyl trimethylammonium bromide, benzalkonium chloride, hexadecyltrimethylammonium bromide.

42. A process for the preparation of nanostructured Sildenafil composition according to claim 34, comprising precipitating nanostructured Sildenafil base or its pharmaceutically acceptable salts or co-crystals from a solution of Sildenafil base or its pharmaceutically acceptable salt and at least one stabilizer or polyelectrolyte or a mixture thereof or additional stabilizer, if desired, in the presence of a pharmaceutically acceptable acid or base, in a continuous flow reactor, preferably in a microfluidic based continuous flow reactor.

43. The process according to claim 42, comprising (1) dissolving Sildenafil base or its pharmaceutically acceptable salt and optionally one or more polyelectrolyte or stabilizer or a mixture thereof in a suitable solvent; (2) adding the formulation from step (1) to a solution comprising one or more polyelectrolytes or stabilizers or a mixture thereof, if desired in the presence of a pharmaceutically acceptable acid or base; and (3) precipitating the formulation from step (2).

44. The process according to claim 43, comprising (1) dissolving Sildenafil base or its pharmaceutically acceptable salt and one or more stabilizer(s) in a suitable solvent; (2) adding the formulation from step (1) to a solution comprising a pharmaceutically acceptable acid or base; and (3) precipitating the formulation from step (2).

45. The process according to claim 42, comprising the use of two different solvents miscible with each other, where Sildenafil base or its pharmaceutically acceptable salt or co-crystal is soluble only in one of them.

46. A pharmaceutical composition comprising a nanostructured composition according to claim 34 together with pharmaceutically acceptable auxiliary materials.

47. The pharmaceutical composition according to claim 46, wherein the composition is formulated into a dosage form of a buccal administration.

48. Use of nanostructured composition according to claim 34 for preparation of a medicament.

49. Use of nanostructured composition according to claim 34 or a pharmaceutical composition comprising the composition according to claim 34 for the treatment of male or female sexual dysfunction and pulmonary arterial hypertension.

50. The use according to claim 49, wherein the composition has
a solubility about 24.5 mg/ml in water,
instantaneous redispersibility in physiological mediums,
enhanced transdermal permeability,
increased absorption in human gastrointestinal tract,
faster onset of action,
for decreasing the dosage used.

51. A method of treating a subject in need for the treatment of male or female sexual dysfunction and pulmonary arterial hypertension by administering to the subject an effective amount of nanostructured composition according to claim 34 or a pharmaceutical composition comprising the composition according to claim 34 together with pharmaceutically acceptable auxiliary materials.

52. The method according to claim 51, wherein the composition has
a solubility about 24.5 mg/ml in water,
instantaneous redispersibility in physiological mediums,
enhanced transdermal permeability,
increased absorption in human gastrointestinal tract,
faster onset of action,
for decreasing the dosage used.
